(19) United States
(12) Patent Application Publication
(10) Pub. No.: US 2025/0186803 A1
(43) Pub. Date: Jun. 12, 2025

(54) MULTIMODAL PROTON THERAPY TREATMENT PLANNING SYSTEM

(71) Applicant: Ion Beam Applications S.A., Louvain-la-Neuve (BE)

(72) Inventors: Rudi LABARBE, Louvain-la-Neuve (BE); Lucian HOTOIU, Louvain-la-Neuve (BE); Arnaud PIN, Louvain-la-Neuve (BE)

(73) Assignee: Ion Beam Applications S.A., Louvain-la-Neuve (BE)

(21) Appl. No.: 19/059,910

(22) Filed: Feb. 21, 2025

Related U.S. Application Data
(63) Continuation of application No. 17/362,471, filed on Jun. 29, 2021.

(30) Foreign Application Priority Data
Jun. 30, 2020 (EP) 20183082.5

(51) Int. Cl. A61N 5/10

(52) U.S. Cl. CPC A61N 5/1031; A61N 5/1042; A61N 5/1077; A61N 2005/1087

(57) ABSTRACT

A system for treating a target tissue with a charged particle beam, wherein the target tissue includes tumoral cells surrounded by or encloses healthy cells of healthy tissue, the system comprising a computer configured to receive a total target dose, receive a fraction number irradiation fractions for treating, receive a maximum healthy fraction dose and a maximum healthy total dose, receive an equivalence coefficient (α), divide the total target dose into the target fraction doses, and determine a ratio of a target fraction dose to be deposited at a conventional deposition rate and a complementary ratio of the target fraction dose to be deposited at a high deposition rate based on an equivalent fraction dose, the maximum healthy fraction dose, a total equivalent dose, and the maximum healthy total dose.

### CROSS-REFERENCE TO RELATED APPLICATIONS

This application is a continuation of U.S. application Ser. No. 17/362,471, filed Jun. 29, 2021, which claims priority to European Patent Application No. 20183082.5, filed Jun. 30, 2020. The entire contents of the aforementioned applications are incorporated herein by reference.

### TECHNICAL FIELD

The present disclosure relates to a treatment planning system (TPS) for generating a plan for treatment with charged particles beams, for instance with proton beams, of a target tissue comprising tumoral cells. The plan fulfils predefined clinical criteria for ensuring that at the end of the treatment, the tumoral cells are destroyed or killed, while preserving the healthy cells adjacent to the tumoral cells. The TPS establishes the plan by combining irradiation sessions or fractions at conventional dose deposition rates (CDR) and/or at ultra-high dose deposition rates (HDR) according to criteria specific to the present disclosure. The plan thus generated ensures an effective treatment of the tumoral cells, with a lower ratio of damages to healthy cells adjacent to the tumoral cells.

### BACKGROUND

Radiation therapy with particles or waves, such as electron beams, protons beams, heavy ions beams, x-rays, γ-rays, and the like, has become an essential tool for treating patients with tumours. Since both tumoral cells and healthy cells are damaged by such radiations, a major challenge in cancer treatment is to define a plan ensuring that the tumoral cells are effectively destroyed or killed, while sparing as much as possible the healthy cells, in particular those adjacent to the tumoral cells. Such plan satisfies multiple, often competing, parameters, and may be complex.

A first criterion a treatment plan must fulfil is to ensure that, at the end of the treatment, a total target dose (DmtT) greater than or equal to a minimum target dose (DmtT0) has been delivered to the tumoral cells forming the target effective for destroying/killing the tumoral cells.

A second criterion to be fulfilled by the treatment plan is to minimize the degradation of the healthy cells adjacent to the tumoral cells. No matter how accurate is the dose deposition in a volume comprising tumoral cells, a radiation beam reaching the volume comprising the tumoral cells almost necessarily crosses healthy cells and delivers thereto a dose also to healthy cells surrounding or included in that volume. Different radiations deposit their energies in different patterns. For example, as shown in FIG. 1, X-rays deposit most of their energy near the level of the skin, and the deposited energy decreases with depth. Healthy tissues located upstream of a target volume of tumoral cells therefore receive a higher dose than the tumoral cells of target volume. By contrast, protons deposit most of their energy close to the end of their beam path, forming a so-called Bragg peak. By superposing a number of beamlets with their respective Bragg peaks staggered in depth, a sum of individual Bragg Peaks (SOBP) can be defined spanning a whole depth of the volume of tumoral cells or of a fraction of that volume. The healthy cells located upstream of the volume of tumoral cells crossed by a proton beam therefore receive a lower dose than the tumoral cells in the volume. Consequently, proton therapy is well suited for depositing high doses in deep seated tumours.

To minimize the degradation of the healthy cells adjacent to the tumoral cells, the total dose (DhTi) received by a healthy cell (i) may not exceed a maximum allowable dose. As the maximum allowable dose a healthy cell can (relatively) safely receive in one session may be substantially lower than the minimum target dose (DmtT0) required to destroy the tumoral cells, the total target dose (DmtT) can be delivered to the tumoral cells in one or more fractions (or sessions).

A treatment plan may therefore include the delivery of a predefined total target dose (DmtT) of radiation to all the tumoral cells in N sessions or fractions j, with N≥1, each fraction j delivering a target fraction dose (Dmtj) to a volume of tumoral cells. If N=1, the whole of the predefined total target dose is delivered in a single session. If N>1, the predefined total target dose is delivered in several fractions, possibly to different volumes of tumoral cells, such that at the end of the N^{th} session, all the tumoral cells have received a cumulated target dose equal to the predefined total target dose (DmtT) for killing tumoral cells. The number N of fractions depends on the nature of the healthy tissues which determines the maximum fraction dose they can safely receive in a fraction. It also depends on the type of radiation used and on the positions of the healthy cells relative to the tumoral cells, as these parameters at least partially determine the dose deposited on healthy cells crossed by a radiation beam aiming at depositing a target fraction dose (Dmtj) onto a volume containing tumoral cells.

It has been observed that the tumoral cells have a longer recovery time to recover from the damages suffered after an irradiation fraction than healthy cells. This allows a treatment plan to span over several fractions distributed over a period of several days and weeks. By ensuring that the healthy cells receive fraction doses low enough to preserve them, the tumoral cells receive a total target dose cumulated over the N fractions at least equal to the minimum target dose (DmtT), and a recovery time between two successive fractions is sufficient to allow substantial recovery of the healthy cells, but not of the tumoral cells, the treatment plan can ensure that at the end of the treatment, the tumoral cells have been destroyed, while sparing the healthy cells.

Maximum fraction dose and maximum total dose acceptable to the healthy cells can be defined relative to a Normal Tissue Complication Probability (NTCP) which defines the probability of a given tissue of developing complications upon exposure to a radiation. Values of boundary doses of radiation yielding a given value of the NTCP for a selection of organs are available in the literature. For example, Kehwar, J Cancer Res Ther, September 2005, 1(3), 168, lists in Table 3, on p. 171, a number of doses yielding a 50% NTCP of developing five years after the treatment symptoms as defined in the last column for a number of organs. The volumes 1/3-3/3 in Table 3 indicate the portion of the total volume of the corresponding organ being irradiated. Alternatively, a medical practitioner can have other sources for determining boundary doses, such as personal experience, or referring to other experimental data.

Historically, treatment plans by radiation therapy included the delivery of radiation doses to the treated cells at a conventional dose deposition rate (CDR) lower than 1 Gy/s. Current radiation therapy facilities deliver dose-rates around 0.1 Gy/s and most clinical protocols involve daily delivery of N target fraction doses (Dmtj) of 2 to 15 Gy cumulated to reach the total target dose (DmtT) which often exceeds the tolerance limit of normal tissues located in the radiation field, thus damaging them together with the tumoral cells. Recently, it has been observed that a same dose (D) had different effects on healthy cells but not on tumoral cells when deposited at conventional dose deposition rates (CDR) or at ultra-high dose deposition rate (HDR); HDR can be one or more orders of magnitude larger than conventional dose deposition rates (CDR) usually applied. Deposition of a charge at ultra-high dose deposition rates (HDR) is also referred to as FLASH-radiotherapy(=FLASH-RT). It has been demonstrated experimentally on animals and on various organs, that ultra-high rate dose deposition at HDR can significantly spare healthy tissues in comparison with conventional deposition of a same dose at CDR and, at the same time, tumoral cells respond same or even better to HDR deposition than to CDR deposition. For example, FLASH-RT reportedly elicits in mice a dramatic decrease of the incidence of lung fibrosis, of memory loss subsequent to brain irradiation, and of necrosis of the small intestine while keeping the anti-tumour efficiency unchanged. Such specific normal tissue sparing has been confirmed in large animals and a patient with cutaneous lymphoma has already been treated with FLASH-RT. Many treatment centers, however, do not dispose of equipment capable of delivering dose at HDR in a time of the order of the ms or s or dispose of equipment that can be modified to deliver HDR only in a very limited field size.

The challenge of generating ever more efficient treatment plans using existing equipment, with enhanced killing of tumoral cells, while sparing as much as possible healthy tissues remains open. The discovery of the differentiated biological reactions of tumoral and healthy cells to radiation doses deposited at ultra-high rates (HDR) provides an additional tool for enhancing the efficacy of the treatment plans. For example, WO2019018813 describes a treatment plan for treating tumours by administering radiations at ultra-high dose deposition rates and a therapeutic agent to a patient with cancer. The treatment plan provides the dual benefits of anti-tumour efficacy plus normal tissue protection when combining therapeutic agents with ultra-high dose deposition rate radiations to treat cancer patients.

The present disclosure proposes a treatment planning system (TPS) for generating a plan for treatment with charged particles beams, for instance, with proton beams, of a target tissue comprising tumoral cells. The plan comprises combining N fractions (N≥1), each fraction including irradiation of volumes including tumoral cells and/or healthy cells at a conventional dose deposition rate (CDR), defined as a dose rate, CDR<1 Gy/s, and/or irradiation of volumes including tumoral cells and/or healthy cells at an ultra-high dose deposition rate (HDR), defined as a dose rate, HDR≥1 Gy/s.

By carefully combining selecting doses deposited at CDR and HDR to tumoral cells and/or to healthy cells, an optimal plan can be achieved with efficient killing of tumoral cells and high sparing of healthy tissues. These and other advantages of the present disclosure are explained more in detail in the following sections.

### SUMMARY

The objectives of the present disclosure have been reached with a treatment planning system (TPS) for generating a plan for treatment with charged particles beams, for instance, with proton beams, of a target tissue comprising tumoral cells enclosed within a peripheral surface, which is surrounded by healthy cells and/or encloses also healthy cells, wherein the healthy cells form healthy tissues. The plan consists of N fractions of irradiation, with N≥1, and the plan comprises fulfilling the following criteria. First, at the end of the N fractions, all tumoral cells of the target tissue (3t) received a total target dose (DmtT) equal to the sum of target fraction doses (Dmtj) received at each fraction (i.e., DmtT=Σ Dmtj), which is at least equal to a minimum target dose (DmtT0) for killing the tumoral cells, i.e., DmtT=Σ Dmtj≥DmtT0. Second, at the end of the N fractions, healthy cells (3hi) of the healthy tissues surrounding or enclosed within the peripheral surface received a total healthy dose (DhTi) equal to the sum of the healthy fraction doses (Dhij) received at each fraction j (i.e., DhTi=Σ Dhij), such that the healthy fraction dose (Dhij) received at each fraction j does not exceed a predefined threshold for preserving the healthy cells at the end of the fraction j, and the total healthy doses (DhTi) received and cumulated at the end of the N fractions does not exceed a predefined threshold for preserving the healthy cells at the end of the treatment of N fractions,

According to the present disclosure, the plan combines N fractions including irradiation of volumes including tumoral cells and/or healthy cells at a conventional dose deposition rate (CDR), defined as a dose rate, CDR<1 Gy/s, and/or irradiation of volumes including tumoral cells and/or healthy cells at an ultra-high dose deposition rate (HDR), defined as a dose rate, HDR≥1 Gy/s, wherein the total target dose (DmtT) delivered to and cumulated in a volume of the target tissue at the end of N fractions may be defined to fulfil criterion (C1) as a sum over the N fractions j, DmtT=Σ Dmtj=Σ(Dctj+Dhtj)≥DmtT0, of target fraction doses (Dmtj) delivered to the target tissues at each fraction. The target fraction dose (Dmtj) delivered during a fraction j may be defined as a sum of, conventional target doses (Dctj) delivered to that volume at conventional dose deposition rates (CDR) during the fraction j, and of high rate target doses (Dhtj) delivered to that volume at ultra-high dose deposition rates (HDR) during the fraction j.

The healthy fraction dose (Dhij) delivered to a volume of healthy cells of a healthy tissue (3i) during a fraction j may be equal to a sum, Dhij=Dchij+Dhhij, of a conventional healthy fraction dose (Dchij) delivered to that volume at a conventional dose deposition rate (CDR), and of a high rate healthy fraction dose (Dhhij) delivered to that volume at ultra-high dose deposition rate (HDR).

The present disclosure provides an equivalent healthy fraction dose (Dmhij) defined as a sum Dmhij=(Dchij+α Dhhij), of the conventional healthy fraction dose (Dchij) and a product of an equivalence coefficient (α) and of the high rate healthy fraction dose (Dhhij), that does not exceed a maximum equivalent healthy fraction dose (Dmh0ij), for preserving the healthy cells after each fraction j (i.e., Dmhij≤Dmh0ij), wherein the maximum equivalent healthy fraction dose (Dmh0ij) is smaller than or equal to a maximum healthy conventional fraction dose (Dch0ij) corresponding to a dose deposited onto a healthy cell at a conventional dose deposition rate (CDR) only and yielding a predefined probability of complication (NTCP0i) of the healthy cell (i.e., Dmh0ij≤Dch0ij).

The present disclosure also includes a total equivalent healthy dose (DmhTi) delivered to and cumulated in a volume of a healthy tissue at the end of N fractions may be defined as a sum over the N fractions j, DmhTi=Σ Dmhij=Σ(Dchij+α Dhhij) with α≤1, or α>0.5, of the equivalent healthy fraction doses (Dmhij), wherein the total equivalent healthy dose (DmhTi) does not exceed a maximum equivalent healthy dose (DmhT0i), for preserving the healthy cells at the end of the N fractions (i.e., DmhTi≤DmhT0i), wherein the maximum equivalent healthy dose (DmhT0i) is smaller than or equal to a maximum healthy conventional dose (DchT0i) corresponding to a dose deposited onto a healthy cell at a conventional dose deposition rate (CDR) only and yielding a predefined probability of complication (NTCP0i) of the healthy cell (3hi) (i.e., DmhT0i≤DchT0i).

The equivalent healthy fraction dose (Dmhij) and the total equivalent healthy dose (DmhTi) are defined as equivalent doses yielding a same or lower probability of complication of a healthy cell (NTCP0i) as if the healthy fraction dose (Dhij) and the total healthy dose (DhTi) had, respectively, been deposited onto the healthy cell at a conventional dose deposition rate (CDR) only.

In an embodiment of the present disclosure, if a volume of a healthy tissue is irradiated during a fraction j at a conventional dose deposition rate (CDR) only, then Dmhij=Dchij≤Dch0ij for that volume at that fraction. Alternatively, at an ultra-high rate (HDR) only, then Dmhij=α Dhhij≤Dch0ij and Dhhij≤Dhh0ij for that volume at that fraction, wherein Dhh0ij is the maximum high rate healthy fraction dose delivered at HDR only at a single fraction j, not to be exceeded for preserving the healthy cells of the healthy tissue (3i), or at both conventional dose deposition rate (CDR) and at ultra-high dose deposition rate (HDR), Dmhij=Dchij+α Dhhij≤Dch0ij and Dhij=Dchij+Dhhij<Dhh0ij.

The equivalence coefficient α is not necessarily constant. For example, α may either be a constant that may be equal to 1/k, wherein k=1.2 to 1.5 (i.e., α=1/k, with k=1.2 to 1.5), and, in some embodiments, α<0.9, or α<0.85, or α<0.7, or α may depend on a HDR-proportion, xH=Dhhij/(Dchij+Dhhij) of doses deposited at ultra-high rate (HDR), and varying between 1/k for xH=1 (i.e., at HDR only), and 1 for xH=0 (i.e., at CDR only).

The values of the boundary doses Dch0ij and Dhh0ij, as well as DchT0i and DhhT0i not to be exceeded to preserve the healthy cells can be determined by different ways. Dch0ij and DchT0i can predefined by a medical practitioner (clinician) or defined in the literature. Dhh0i and DhhT0i, on the other hand, can be either predefined by a medical practitioner or defined in the literature for given values of the HDR, or respectively defined as Dhh0ij=k Dch0ij and Dhh0Ti=k DchT0i, for given values of the HDR, wherein k is comprised between 1.2 and 1.5 (i.e., k=1.2 to 1.5), or defined by a pre-established curve of a Normal Tissue Complication Probability (NTCP) for the healthy tissue as a function of dose and dose rate, and wherein Dhh0ij and DhhT0i are defined as the doses delivered at HDR only having same NTCP as the doses Dch0ij and as DchT0i, respectively, delivered at CDR only.

For example, the maximum healthy conventional dose (DchT0i) and the maximum healthy high-rate dose (DhhT0i) at the end of the N fractions may be defined as follows for a selection of healthy tissues

| Healthy tissue (3hi) | DchT0i (Gy) | DhhT0i (Gy) |
|---|---|---|
| brain | 60 | 90 |
| heart | 50 | 75 |
| larynx | 80 | 120 |
| Liver | 40 | 60 |
| Lung | 25 | 38 |
| Stomach | 65 | 98 |
| Eye lens | 18 | 27 |

In one embodiment, all healthy tissues located upstream from the peripheral surface may be irradiated at conventional dose deposition rates only during the N fractions, and wherein, the healthy fraction dose (Dhij) deposited in said healthy tissues at each fraction j (is equal to the conventional healthy fraction dose (Dchij) and) does not exceed the maximum healthy conventional fraction dose (Dch0ij), and the total healthy dose (DhTi) deposited in said healthy tissues at the end of the N fractions (is equal to the conventional healthy dose (Dchi) and) does not exceed the maximum healthy conventional dose (Dch0Ti), wherein a healthy cell is located upstream of the peripheral surface when a charged particles beam defined in the TPS traverses said healthy cell before penetrating into the peripheral surface.

In an embodiment, all healthy tissues located downstream of the peripheral surface may be irradiated at ultra-high dose deposition rates only. The healthy fraction dose (Dhij) deposited in said healthy tissues at each fraction j (is equal to the high-rate healthy fraction dose (Dhhij) and) does not exceed the maximum high rate healthy fraction dose (Dhh0ij), and the total healthy dose (DhTi) deposited in said healthy tissues at the end of the N fractions (is equal to the high-rate healthy dose (DhhTi) and) does not exceed the maximum healthy conventional dose (Dch0Ti). In some embodiments, a healthy cell may be located downstream of the peripheral surface when a charged particles beam traverses said healthy cell only after exiting the peripheral surface.

In an embodiment, one or more of the fractions of the plan include the following steps carried out in any sequential order. A first step of delivering a conventional dose at a conventional dose deposition rate (CDR) with a first charged particles beam or group of charged particles beams parallel to a first direction each formed by various beamlets, such that, a conventional target fraction dose (Dctj) is delivered at each or some of the one or more fractions to at least 90%; or, in some embodiments, at least 95%, or at least 99% of the tumoral cells of the target tissue (3t), and a conventional healthy fraction dose (Dchi) lower than the maximum healthy conventional fraction dose (Dch0ij) is delivered at each or some of the one or more fractions to the healthy tissues (3hi) surrounding and enclosed within the peripheral surface (i.e., Dchij<Dch0ij). A second step may include delivering a high rate dose at an ultra-high dose deposition rate (HDR) with a second charged particles beam or group of charged particles beams parallel to a second direction same as or different from the first direction, such that, a high rate target fraction dose (Dhtj) is delivered at each or some of the one or more fractions to the target tissue such that 100% of the tumoral cells of the target tissue have received a fraction dose at conventional and/or at ultra-high rates (CDR, HDR), and a high rate healthy fraction dose (Dhhij) is delivered at each or some of the one or more fractions to the healthy tissues surrounding and within the peripheral surface, such that the equivalent healthy fraction dose (Dmhij=Dchij+α Dhhij) delivered at both conventional and ultra-high rates (CDR, HDR) to all healthy cells, does not exceed the maximum conventional healthy fraction dose (Dmh0ij) (i.e. Dmhij≤Dch0ij).

The second charged particles beam or group of charged particles beams may be selected such as not to overlap with the first charged particles beam or group of charged particles beams in any healthy tissue, surrounding or within the peripheral surface.

In an alternative embodiment, the plan includes a single charged particles beam (PB) or group of beams parallel to a first direction formed by beamlets, such that, a conventional target fraction dose (Dctj) is delivered to at least 90%, or, in some embodiments, at least 95%, or at least 99% of the tumoral cells of the target tissue, a conventional healthy fraction dose (Dchij) lower than the maximum healthy conventional fraction dose (Dch0ij) is delivered to the healthy tissues (3hi) surrounding and enclosed within the peripheral surface (i.e., Dchij<Dch0ij), a high rate target fraction dose (Dhtj) is delivered at each or some of the one or more fractions to the target tissue such that 100% of the tumoral cells of the target tissue have received a fraction dose at conventional and/or at ultra-high rates (CDR, HDR), and a high rate healthy fraction dose (Dhhij) is delivered at each or some of the one or more fractions to the healthy tissues surrounding and within the peripheral surface, such that the equivalent healthy fraction dose (Dmhij=Dchij+α Dhhij) delivered at both conventional and ultra-high rates (CDR, HDR) to healthy cells does not exceed the maximum conventional healthy fraction dose (Dch0ij) (i.e. Dmhij≤Dch0ij).

The peripheral surface may comprise an uncertain volume defining an uncertain zone, defined as a zone comprising both tumoral and healthy cells intermixed at different ratios. In this case, the TPS can comprise the delivery to an entirety of the uncertain volume at the end of N fractions of a total healthy dose (DhTi) such that the total healthy dose (DhTi=DchTi+DhhTi) is larger than the minimum target dose (DmtT0), and lower than the maximum high rate healthy dose (DhhT0i) (i.e., DmtT0<DhTi<DhhT0i), and a total equivalent healthy dose (DmhTi=DchTi+α DhhTi) delivered at the end of the N fractions is lower than the maximum healthy conventional dose (DchT0i) of the healthy tissue (3hi) present in the uncertain zone (i.e., DmhTi<DchT0i), and an equivalent healthy fraction dose (Dmhij=Dchij+α Dhhij) delivered at each fraction j is lower than the maximum healthy conventional fraction dose (DchT0ij) of the healthy tissue (3hi) present in the uncertain zone, (i.e., DmhTi<DchT0i).

The TPS of the present disclosure may include a proton beam as the charged particle beam, which can be delivered in single- or double-scattering mode, or by pencil beam scanning (PBS).

### BRIEF DESCRIPTION OF THE FIGURES

FIG. 1(a) shows the energy deposition onto tissues as a function of depth (z) of penetration, for X-rays (XR), and for proton beamlets, adding up to form a sum of individual Bragg Peaks (SOBP) spanning over the whole depth of the tumour (3), 1(b) shows a cut parallel to a proton beam direction crossing a target tissue comprising tumoral cells, and 1(c) shows the corresponding SOBP.

FIG. 2(a) illustrates a target tissue (3t) comprising tumoral cells enclosed within a peripheral surface, which is surrounded by healthy cells (3hi) and/or encloses also healthy cells (3hi), 2(b) illustrates a cut along a plane (x, z) parallel to the charged particles beam of the target tissue of FIG. 2(a).

FIG. 3 shows a portion of a treatment plan referring to the healthy tissues (3hi) and divided in N fractions, each fraction comprising delivery of a predefined dose (Dhij) at conventional and/or ultra-high rate (CDR, HDR) cumulating to a total equivalent healthy dose (DmhTi) at the end of the N^{th} fraction.

FIG. 4(a) shows iso-NTCP-lines as a function of dose (D) deposited into a healthy tissue and of the dose deposition rate (DR), 4(b) NTCP as a function of dose (D) deposited into a healthy tissue at CDR (white circles) and at HDR (black circles), and 4(c) shows the principle of the equivalent healthy fraction dose (Dmhij=Dchij+α Dhhij).

FIG. 5 illustrates (a) Dmhij(=Dchij+α Dhhij) as a function of Dhij(=Dchij+Dhhij) for a dose deposition only at CDR(=“Dhhij=0 (CDR)”), only at HDR(=“Dchij=0 (HDR)”), and a linear(=“(1)”) and non-linear(=“(4)”) assumptions for a mixed deposition mode with a dose Dch1ij deposited at CDR, and the rest of the dose deposited at HDR, (b) Dh0ij as a function of xH, for four options of α=f(xH), as represented in FIG. 5(c).

FIG. 6 shows a treatment plan including a dose (D) deposition profile as a function of depth of penetration (z), as well as a dose deposition rate (DR) profile as function of the same depth of deposition.

FIG. 7 shows the principle of the equivalent healthy dose (DmhTi=DchTi+α DhhTi).

### DETAILED DESCRIPTION

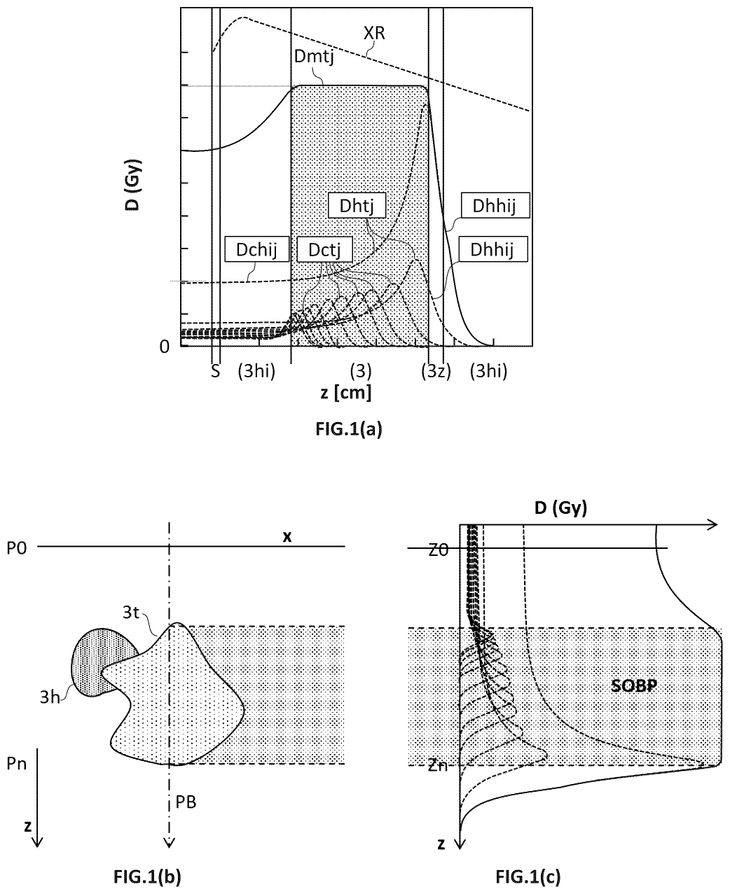

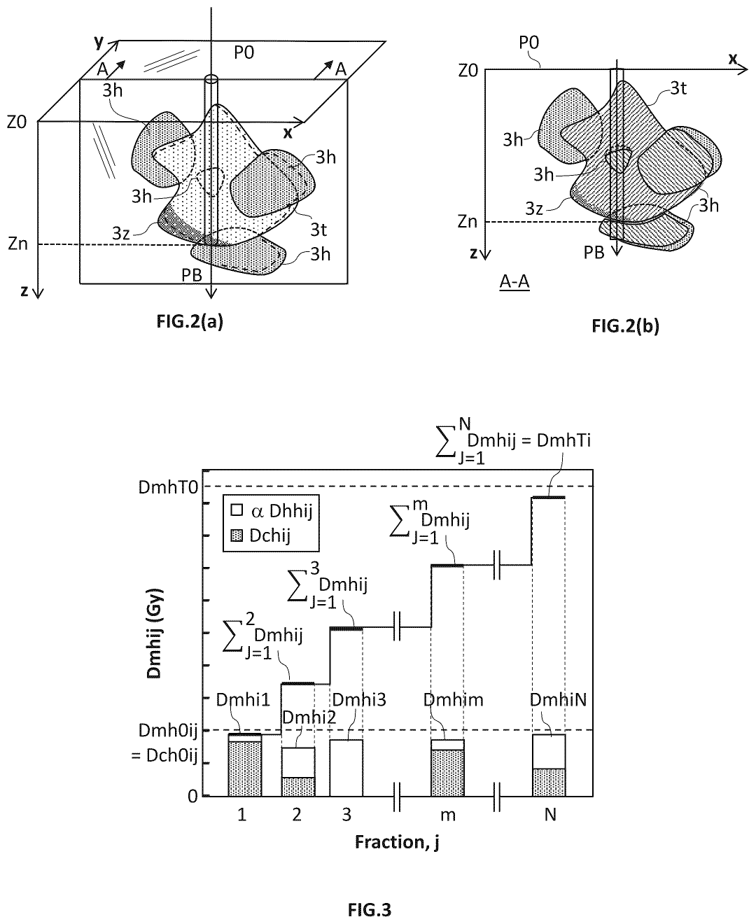

The present disclosure concerns a treatment planning system for generating a plan for treatment with charged particles beams of a target tissue (3t). Examples of target tissues are illustrated in FIGS. 1(b), 2(a), and 2(b), comprising tumoral cells enclosed within a peripheral surface, which is surrounded by healthy cells and/or encloses also healthy cells, wherein the healthy cells form healthy tissues (3hi) wherein the subscript i designates the type of healthy tissues (3hi). Because of the complexity of the multi-parameter treatment to be planned, the TPS may comprise a computer or processor configured for optimizing the nature and number of beams or beamlets required for fulfilling predefined criteria. The nature of the beam includes the type of charged particles, the intensity of the beam, the doses to be deposited, the dose deposition rates, the direction of the beams, the number and frequency of fractions, and the like. Because of the dose deposition profile including a Bragg peak as illustrated in FIGS. 1(a) and 1(c), the charged particles may be protons. The charged particle beam can be delivered in single- or double-scattering mode, or by pencil beam scanning (PBS).

As shown in FIG. 3, the plan consists of N fractions of irradiation, with N≥1, the TPS may optimize the nature and number of beam irradiations fulfilling the following criteria. At the end of the N fractions, all tumoral cells of the target tissue (3t) must have received a total target dose (DmtT) equal to the sum of target fraction doses (Dmtj) received at each fraction (i.e., DmtT=Σ Dmtj), which is at least equal to a minimum target dose (DmtT0) for killing the tumoral cells, i.e., DmtT=Σ Dmtj≥DmtT0. At the end of the N fractions, all healthy cells (3hi) of the healthy tissues surrounding or enclosed within the peripheral surface must have received a total healthy dose (DhTi) equal to the sum of the healthy fraction doses (Dhij) received at each fraction j (i.e., DhTi=Σ Dhij), such that, the healthy fraction dose (Dhij) received at each fraction j does not exceed a predefined threshold for preserving the healthy cells at the end of the fraction j, and the total healthy doses (DhTi) received and cumulated at the end of the N fractions does not exceed a predefined threshold for preserving the healthy cells at the end of the treatment of N fractions

While the FLASH effect is now commonly acknowledged by the persons skilled in the art, the chemical mechanisms underlying the FLASH effect are still elusive. Several different theories have been advanced. For example, it has been proposed that the ultra-high dose deposition rate FLASH-RT significantly reduces the killing of cells circulating in the bloodstream, suggesting that the threshold dose deposition rate separating CDR and HDR could depend on the blood circulation time for one cycle. For humans, a threshold dose deposition rate of about 1 Gy/s has been reported for the FLASH effect to appear. It has also been proposed that organic peroxyl radicals ROO· formed by addition of O₂ to primary carbon-centered radicals could play a major role in radio-induced complications. In another example, it has also been reported that the competition between the radio-induced oxygen depletion and the oxygen rediffusion from capillaries could explain the FLASH effect. The present disclosure is not bound by any one of these or other theories and is based on the experimental evidence that the FLASH effect actually exists. The present disclosure was developed based on the following observations.

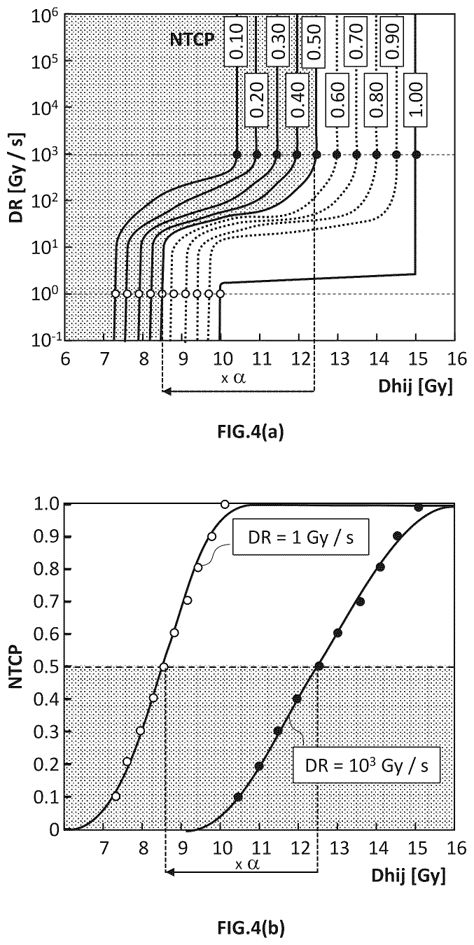

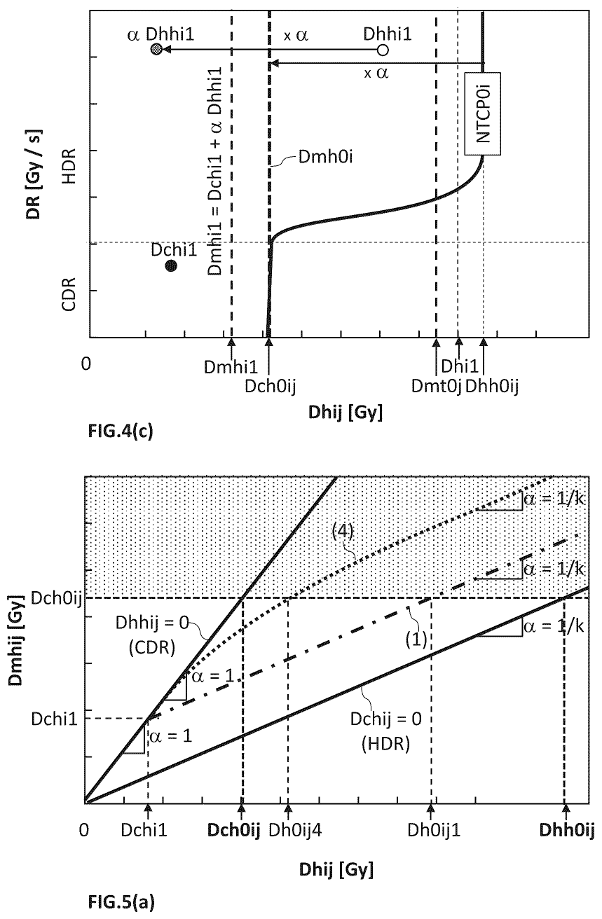

First, tumoral cells are killed similarly, independently of the dose deposition rate (DR). A predefined Normal Tissue Complication Probability (NTCP0i) of a healthy tissue (3hi) not to be exceeded is reached at lower doses deposited at conventional dose deposition rates (CDR) than it is at ultra-high dose deposition rates (HDR), as shown in FIGS. 4(a) to 4(c). Tumoral cells in target tissues (3t) have a longer recovery time than healthy tissues exposed to a same radiation dose, regardless of the dose deposition rate.

In order to fulfil the foregoing criteria and as illustrated in FIG. 3, the TPS of the present disclosure may combine N fractions including, irradiation of volumes including tumoral cells and/or healthy cells at a conventional dose deposition rate (CDR), defined as a dose rate, CDR<1 Gy/s, and/or irradiation of volumes including tumoral cells and/or healthy cells at an ultra-high dose deposition rate (HDR), defined as a dose rate, HDR≥1 Gy/s.

Based on observations discussed herein, the total target dose (DmtT) delivered to and cumulated in a volume of the target tissue (3t) at the end of N fractions may be defined as a sum over the N fractions j of total target fraction doses (Dmtj) delivered to the target tissues (3t) at each fraction. The target fraction dose (Dmtj) delivered during a fraction j may be defined as a sum of conventional target doses (Dctj) delivered to that volume at conventional dose deposition rates (CDR) during the fraction j, and of high rate target doses (Dhtj) delivered to that volume at ultra-high dose deposition rates (HDR) during the fraction j. This may be expressed as follows: DmtT=Σ Dmtj=Σ(Dctj+Dhtj)≥DmtT0.

The healthy fraction dose (Dhij) delivered to a volume of healthy cells of a healthy tissue (3i) during a fraction j may be equal to a sum, Dhij=Dchij+Dhhij, of a conventional healthy fraction dose (Dchij) delivered to that volume at a conventional dose deposition rate (CDR), and of a high rate healthy fraction dose (Dhhij) delivered to that volume at ultra-high dose deposition rate (HDR).

Based on observation that a given NTCP0i not to be exceeded of a healthy cell is reached at lower conventional healthy doses (Dchij) deposited at CDR than it is at HDR, an equivalent healthy fraction dose (Dmhij) may be defined as a sum Dmhij=Dchij+α Dhhij, of the conventional healthy fraction dose (Dchij) and a product of an equivalence coefficient (α) and of the high rate healthy fraction dose (Dhhij),

The equivalent healthy fraction dose (Dmhij) may be defined as an equivalent dose yielding a same or lower probability of complication of a healthy cell (3hi) (NTCP0) as if the healthy fraction dose (Dhij) had been deposited onto the healthy cell at a conventional dose deposition rate (CDR) only. This definition is a direct consequence of observation (O2) supra.

In order to fulfil the criterion (C2.1) supra, the equivalent healthy fraction dose (Dmhij) must not exceed a maximum equivalent healthy fraction dose (Dmh0ij), for preserving the healthy cells after each fraction j (i.e., Dmhij≤Dmh0ij). The maximum equivalent healthy fraction dose (Dmh0ij) is smaller than or equal to a maximum conventional healthy fraction dose (Dch0ij) corresponding to a dose deposited onto a healthy cell at a conventional dose deposition rate (CDR) only and yielding a predefined probability of complication (NTCP0i) of the healthy cell (3hi) (i.e., Dmh0ij≤Dch0ij). Dmh0ij can be smaller than Dch0ij to allow for a safety margin in case the equivalence coefficient α for a specific healthy tissue (3hi) is not defined with sufficient certainty or accuracy.

In order to fulfil the criterion (C2.2) supra, a total equivalent healthy dose (DmhTi) delivered to and cumulated in a volume of a healthy tissue (3hi) at the end of N fractions may be defined as a sum over the N fractions j, DmhTi=Σ Dmhij=Σ(Dchij+α Dhhij) with α≤1, or, in some embodiments, α>0.5, of the equivalent healthy fraction doses (Dmhij). The total equivalent healthy dose (DmhTi) must not exceed a maximum equivalent healthy dose (DmhT0i), for preserving the healthy cells at the end of the N fractions (i.e., DmhTi≤DmhT0i).

The total equivalent healthy dose (DmhTi) may be defined as an equivalent dose yielding a same or lower probability of complication of a healthy cell (3hi) (NTCP0) as if the total healthy dose (DhTi) had been deposited onto the healthy cell at a conventional dose deposition rate (CDR) only. This definition is a direct consequence of observation (O2) supra. The maximum equivalent healthy dose (DmhT0i) is smaller than or equal to a maximum conventional healthy dose (DchT0i) corresponding to a dose deposited onto a healthy cell at a conventional dose deposition rate (CDR) only and yielding a predefined probability of complication (NTCP0i) of the healthy cell (3hi) (i.e., DmhT0i≤DchT0i). Here again, DmhT0i can be smaller than DchT0i to allow for a safety margin in case the equivalence coefficient α for a specific healthy tissue (3hi) is not defined with sufficient certainty or accuracy.

It has been observed that healthy dose Dhij(NTCP0i) may be a function of dose deposition rate (DR). FIGS. 4(a) to 4(c) illustrate graphically the dependence on the dose deposition rate (DR) of the healthy dose (Dhij) required for reaching a given NTCP for a given healthy tissue (3hi) during a fraction j of the treatment. Note that the expression “healthy dose” is used herein to refer to “dose delivered to a healthy cell,” and is not to be understood as a dose which is beneficial to the health of a cell. According to the criteria herein, the “healthy doses” may be such as to limit as much as possible the damages to the healthy cells traversed by beams of charged particles while, at the same time the tumoral cells of the target tissue (3t) are killed by the same beams.

FIG. 4(a) shows iso-NTCP lines in a plot of the dose deposition rate (DR) as a function of healthy dose (Dhij) for values of NTPC comprised between 10% and 100% probability. Assuming the value NTCP0i of the NTCP not to be exceeded during a fraction j of the treatment is 50% probability of complication, then doses left of the iso-NTCP line labelled (0.5) represented by a shaded area may be deposited at corresponding dose deposition rates. It can be seen that the dose (D) defining the iso-NTCP line=50% strongly depends on the rate at which the dose is being deposited.

FIG. 4(b) is a direct consequence of FIG. 4(a), wherein the white dots aligned at the ordinate DR=1 Gy/s of FIG. 4(a) are reported in FIG. 4(b) showing the doses (Dhij) required for reaching a given NTCP at said dose rate, yielding a sigmoid curve of white dots. The same exercise was repeated with the black dots aligned at the ordinate DR=10³ Gy/s of FIG. 4(a) which are reported in FIG. 4(b) yielding the sigmoid curve of black dots. Taking NTCP0i=50% as in FIG. 4(a), yields a corresponding shaded area indicative of the doses which can be deposited onto healthy cells of a healthy tissue (3hi) at these two deposition rates.

The iso-NTCP lines in FIGS. 4(a) and 4(b) define the doses (Dhij) required to reach a given NTCP when the whole of each dose is delivered at the corresponding dose deposition rate (DR). In other words, in an embodiment of the disclosure, if a volume of a healthy tissue (3hi) is irradiated during a fraction j at a conventional dose deposition rate (CDR) only, then the equivalent healthy fraction dose (Dmhij) is equal to the conventional healthy fraction dose (Dchij) (i.e., Dmhij=Dchij), which may be smaller than or equal to the maximum conventional healthy fraction dose (Dch0ij) (i.e., Dmhij=Dchij≤Dch0ij) for that volume at that fraction j. Alternative, if at an ultra-high rate (HDR) only, then the equivalent healthy fraction dose (Dmhij) is equal to the product of the equivalence coefficient (α) and of the high rate healthy fraction dose (Dhhij) (i.e., Dmhij=α Dhhij), which must be smaller than or equal to the maximum conventional healthy fraction dose (Dchij) (i.e., Dmhij=α Dhhij≤Dch0ij) and the high rate healthy fraction dose (Dhhij) must be smaller than or equal to a maximum high rate healthy fraction dose (Dhh0ij) (i.e., Dhhij≤Dhh0ij) for that volume at that fraction, wherein Dhh0ij is the maximum high rate healthy fraction dose delivered at HDR only at a single fraction j, not to be exceeded for preserving the healthy cells of the healthy tissue (3i).

The present disclosure also provides fractions j combining doses deposited at both conventional dose deposition rates (CDR) and ultra-high dose deposition rates (HDR). At both conventional dose deposition rate (CDR) and at ultra-high dose deposition rate (HDR), the equivalent healthy fraction dose (Dmhij) is equal to the sum of the conventional healthy fraction dose (Dchij) and of the product of the equivalence coefficient α and of the high rate healthy fraction dose (Dhhij) (i.e., Dmhij=Dchij+α Dhhij), which must be smaller than or equal to the maximum conventional healthy fraction dose (Dch0ij) (i.e., Dmhij=Dchij+α Dhhij≤Dch0ij).

This is illustrated in FIG. 4(c), wherein the black dot labelled “Dchi1” is a dose deposited onto healthy cells at CDR and the white dot labelled ‘Dhhi1” is a dose deposited at HDR onto the same healthy cells during a same fraction j=1. Note that in the example of FIG. 4(c), Dchi1<Dmhi1<Dch0ij<Dhhi1<Dhi1<Dhh0ij. Strictly speaking, the present disclosure is satisfied as long as Dmhij<Dch0ij, and Dhij is necessarily smaller than Dhh0ij. Note that Dhhij is necessarily smaller than Dhij, or it can be equal to Dhij if and only if Dchij=0 (i.e., deposition at HDR only). The healthy fraction dose (Dhij) can be greater or smaller than or equal to Dch0i, as long as the equivalent healthy fraction dose (Dmhij) is smaller than or equal to Dch0i. Contrary to what could be intuitively expected, the healthy fraction dose Dhij=Dchij+Dhhij can be substantially larger than the maximum conventional healthy fraction dose (Dch0ij) as is the case in the example of FIG. 4(c).

The maximum conventional healthy fraction dose (Dch0ij) is generally the predefined threshold value given by a clinician as criterion for establishing a treatment plan which preserves the healthy cells at the end of the fraction j(=criterion (C2.1)). Though greater than Dch0ij, the healthy fraction dose (Dhi1) surprisingly fulfils the criterion (C2.1). This is made possible because the equivalent healthy fraction dose (“α Dhhi1”) of the dose (Dhhi1) deposited at ultra-high rate only is only a fraction defined by the equivalence coefficient α of the high rate healthy fraction dose (Dhhi1) actually deposited at HDR. The corresponding equivalent healthy fraction dose is indicated FIG. 4(c) with a shaded dot labelled “α Dhhi1” and is obtained by multiplying the dose Dhhi1 by the equivalence coefficient α. The equivalent healthy fraction dose (Dmhi1) is indicated by a dashed line and is simply the sum, Dmhi1=Dchi1+α Dhhi1. It can be seen that in the example of FIG. 4(c), the equivalent healthy fraction dose (Dmhi1) is smaller than the maximum conventional healthy fraction dose (Dch0ij) thus fulfilling the criterion (C2.1) for the fraction j=1 illustrated therein. To preserve the healthy tissues (3i), care must be taken that the total healthy dose (Dhi1) does not exceed a maximum healthy fraction dose (Dh0ij) discussed in continuation.

In many cases as illustrated in FIG. 4(c), a minimum target fraction dose (Dmt0j) required to kill tumoral cells in a volume irradiated during a fraction j may be comprised between Dch0ij and Dhh0ij. An advantage of the present disclosure is that a volume comprising both tumoral cells and healthy cells may be radiation treated during a fraction j=1 by deposition of a given fraction dose (Dhi1=Dmt1) at combined deposition rates of CDR and HDR, killing the tumoral cells, because the target fraction dose (Dmt1) is greater than the minimum target fraction dose (Dmt0j) and, at the same time, preserving the healthy cells comprised in the irradiated volume, because the healthy fraction dose (Dhi1) is such that the total equivalent healthy fraction dose (Dmhi1) is smaller than the maximum healthy conventional fraction dose (Dch0ij). In summary, a volume comprising both tumoral cells and healthy cells may be treated by deposition of a fraction dose (Dhij=Dmtj) at both CDR and HDR provided that Dchij+Dhhij=Dhij=Dmtj>Dmt0j, to kill the tumoral cells in the volume, and Dchij+α Dhhij=Dmhij≤Dch0ij, to preserve the healthy cells in the volume.

It is clear to a person skilled in the art, that the foregoing discussion of FIG. 4(c) for a fraction j=1 applies mutatis mutandis to any fraction j (i.e., the subscript “1” can be replaced by “j”).

### Equivalence Coefficient α

The value of the equivalence coefficient α can be determined for dose depositions at ultra-high dose deposition rates (HDR) by establishing NTCP curves as a function of dose (Dhij) and dose deposition rate (DR) as illustrated for example in FIGS. 4(a) to 4(c). At sufficiently high dose deposition rates, wherein the dose (Dhh0ij) yielding a predefined NTCP0i becomes independent of the dose rate the factor a becomes substantially constant (i.e., the right-hand vertical portion of the solid curve in FIGS. 4(a), 4(c), and 7), and may be defined as being equal to 1/k, wherein k=1.2 to 1.5 (i.e., α=1/k, with k=1.2 to 1.5), and α<0.9, or, in some embodiments, α<0.85, or α<0.7.

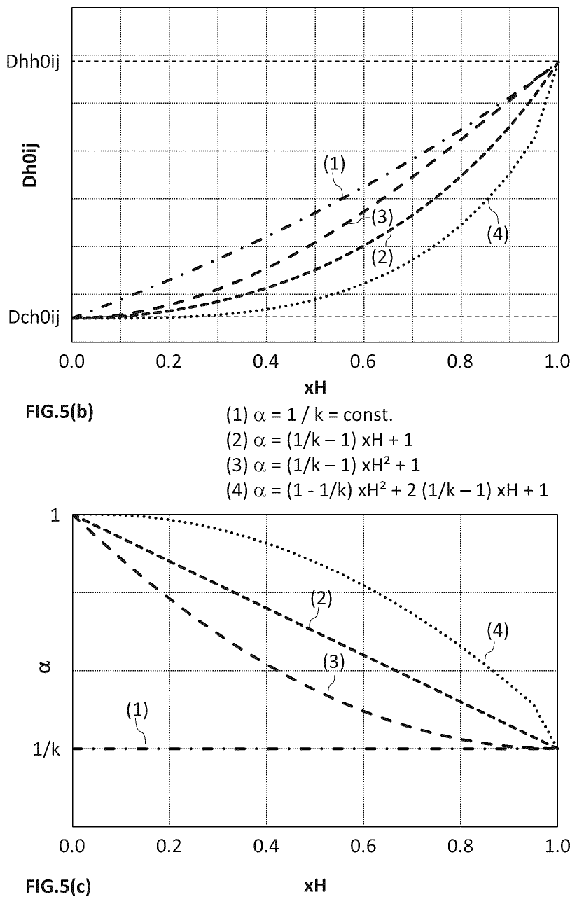

FIG. 5(a) illustrates various examples of the relationship between the equivalent healthy fraction dose (Dmhij) and the healthy fraction dose (Dhij). The solid lines show the relationship for deposition at CDR only, labelled “Dhhij=0 (CDR)”, and at HDR only, labelled “Dchij=0 (HDR)”. Considering that Dmhij=Dchij+α Dhhij, and that Dhij=Dchij+Dhhij, it follows that, at CDR only, Dhhij=0 and therefore Dmhij=Dhij, defining the straight line of slope 1 labelled “Dhhij=0 (CDR)” of FIG. 5(a), which intercepts the horizontal dashed line Dmhij=Dch0ij not to be exceeded to preserve the healthy tissues (3hi) at a value of Dhij=Dch0ij, and at HDR only, Dchij=0 and therefore Dmhij=α Dhhij, defining the straight line of slope α=1/k labelled “Dchij=0 (HDR)” of FIG. 5(a), which intercepts the horizontal dashed line Dmhij=Dch0ij not to be exceeded to preserve the healthy tissues (3hi) at a value of Dhij=Dhh0ij.

In a treatment plan comprising dose deposition at both conventional and ultra-high rates (CDR, HDR), the relationship between Dmhij and Dhij is comprised between the two straight lines of slope 1(=“Dhhij=0 (CDR)”) and of slope α=1/k(=“Dchij=0 (HDR)”) of FIG. 5(a). Fixing the conventional healthy dose (Dchij) deposited in a volume during a treatment fraction to Dch1i (i.e., Dchij=Dch1i=const.), then Dchij=Dch1i+α Dhhij, wherein Dhhij is free to vary from 0(=CDR only) to any positive value. In case the whole dose is deposited at CDR only (i.e., Dhhij=0), then Dchij=Dhij=Dch1i, which marks the starting point of the curve relating Dmhij and Dhij.

Defining a HDR proportion, xH=Dhhij/(Dchij+Dhhij), ranging from xH=0, defining deposition at CDR only, to xH=1, defining deposition at HDR only and defining a proportion of the healthy fraction dose (Dhij) which is deposited at HDR, Dmhij=Dch1i+α Dhhij can also be expressed as a function of Dhij as, Dmhij=(1+xH(α−1))Dhij. The maximum healthy dose (Dh0ij) which can be deposited onto healthy cells of a healthy tissue (3hi) as a function of xH can be determined by equating Dmhij=Dch0ij=(1+xH(α−1))Dh0ij, which is represented graphically in FIG. 5(b), for different values of α, depending on whether or not and how it varies as a function of xH, as illustrated in FIG. 5(c).

In one embodiment of the disclosure labelled (1) in FIG. 5(c), the equivalence coefficient α is independent of the HDR proportion, xH=Dhhij/(Dchij+Dhhij). The HDR proportion (xH) can also be expressed as, xH=1−Dch1i/Dhij. It follows that if the equivalence coefficient α is independent of xH, it is also independent of Dhij. The relation Dchij=Dch1i+α Dhhij therefore defines a straight line of slope α=1/k starting at the point Dmhij=Dhij=Dch1i. This embodiment is illustrated in FIGS. 5(a) and 5(b) with the corresponding dashed lines labelled “(1)” showing linear relationships between Dmhij and Dhij starting at the point Dmhij=Dhij=Dchi1 (cf. FIG. 5(a)) and between a non-linear relationship between Dch0ij and xH (cf. FIG. 5(b)).

In an alternative embodiment, the equivalence coefficient α depends on the HDR-proportion, xH=Dhhij/(Dchij+Dhhij) of doses deposited at ultra-high rate (HDR), and varies between α=1/k for xH=1 (i.e., at HDR only), and α=1 for xH=0 (i.e., at CDR only). Three such embodiments are illustrated in FIG. 5(c) as examples. The short-dashed line (2) defining a linear relationship between α and xH, α=(1/k−1)xH+1; this embodiment is illustrated in FIG. 5(b) with the corresponding short-dashed line (2). The long-dashed line (3) defining a polynomial relationship between α and xH, α=(1/k−1)xH²+1 corresponds to an embodiment illustrated in FIG. 5(b) with the corresponding long-dashed line (3). The dotted line (4) defining a second polynomial relationship between α and xH, α=(1−1/k)xH²+2(1/k−1)xH+1 corresponds to an embodiment illustrated in FIGS. 5(a) and 5(b) with the corresponding dotted lines labelled “(4)”. FIG. 5(a) shows a non-linear relationship between Dmhij and Dhij with a conventional healthy fraction dose Dchij=Dch1ij=constant, starting at Dhij=Dmhij=Dch1ij and intercepting at a value of Dhij=Dhi0ij(4) the predefined threshold (Dch0ij) represented by the horizontal dashed line at Dmhij=Dch0ij not to be exceeded to preserve the healthy tissues (3hi).

Whether the equivalence coefficient α depends or not on the HDR-proportion xH is mostly dependent on the type of healthy tissue (3hi) concerned and a skilled person knows where to find or how to assess this information. The abscissa (Dhij) of FIG. 5(a) shows how doses (Dhij) substantially higher than the predefined threshold defined by a clinician with the maximum conventional healthy fraction dose (Dch0ij) can be irradiated without exceeding the predefined NTCP0i value specified for preserving the integrity of a given healthy tissue (3hi), by using HDR, either alone or in combination with CDR. FIG. 5(b) shows how the maximum healthy dose (Dh0ij) increases above Dch0ij with increasing values of xH (i.e., with increasing contribution of Dhhij in the radiation treatment). This is of interest for the following reasons. Although a treatment plan aims at reducing the doses deposited onto healthy cells relative to the doses deposited onto tumoral cells, in practice, this is not always feasible. For example, if an irradiated volume comprises both tumoral and healthy cells, then a substantially same dose is deposited onto both tumoral and healthy cells, such that the target fraction dose (Dmti) is substantially equal to the healthy fraction dose (Dhij) (i.e., Dmtj≃Dhij). In most cases, the minimum target fraction dose (Dmt0j) required for killing tumoral cells in a volume in a fraction j is higher than the maximum conventional healthy fraction dose (Dch0ij) (i.e., Dmt0j>Dch0ij). These two competing conditions (viz., Dmtj≃Dhij and Dmt0j>Dch0ij) make it very difficult to kill tumoral cells while sparing the healthy cells comprised within the volume within the predefined value of the NTCP0i (i.e., if Dmt0j>Dch0ij→problem). Since the tumoral cells kill rate of a dose is substantially independent of the dose deposition rate (cf. Observation (O1) supra), and since the NTCP of healthy cells at the same dose decreases with higher deposition rates (cf. Observation (O2) supra), it is possible to deposit a target fraction dose (Dmti) higher than the maximum conventional healthy fraction dose (Dch0ij), yet still keeping within the predefined value of NTCP0i, required for sparing the healthy tissues (3hi).

The window defining the doses which can be deposited in a volume in a fraction j yielding a value of the NTCP≤NTCP0i spans between Dch0ij and Dhh0ij. If the whole dose (Dmti) is deposited at CDR only, the target fraction dose (Dmtj) cannot exceed the maximum conventional healthy fraction dose (Dch0ij) which defines the lowest boundary of the window. This situation is representative of the state of the art, with the problem discussed supra in case Dmt0j>Dch0ij. By combining depositions at both CDR and HDR, the maximum healthy dose (Dh0ij) increases with increasing HDR-proportion xH as illustrated in FIG. 5(b), depending on the dependency of α vs xH.

The highest allowable dose (Dhij) which can safely be deposited onto the healthy tissue in a fraction j is the maximum high rate healthy fraction dose (Dhhij) which can be deposited if the whole target fraction dose (Dmtj) is deposited at HDR (i.e., at xH=1). If the prescribed target fraction dose (Dmtj) required for killing tumoral cells in a volume in a fraction j increases, it becomes more difficult to deposit the whole dose (Dmti) at HDR only. This is mainly due to limitations of the equipment which may be unable to deposit more than a given dose in a given time of the order of the ms to s. By combining CDR and HDR to deposit the whole target fraction dose (Dmtj) in one fraction j, a treatment plan implementable in existing equipment can be generated wherein the total dose (Dmtj≃Dhij) deposited into the volume can be greater than or equal to the minimum target fraction dose (Dmt0j) required for killing tumoral cells in the volume in the fraction j.

If the equivalence coefficient α=1/k≠f(xH) and is constant, illustrated in FIGS. 5(a) to 5(c) with the mixed straight line labelled “(1)”, then a dose up to a maximum value (Dhi0ij(1)) can be deposited onto healthy cells within the predefined NTCP0i. If the equivalence coefficient α=f(xH) with α=1 for Dhhij=0 and α=1/k for Dhhij=1, illustrated in FIG. 5(a) with the dotted curved line labelled ‘(4)”, a dose up to a maximum value (Dhi0ij(4)) can be deposited onto healthy cells within the predefined NTCP0i. The maximum dose values which can be deposited into a volume comprising healthy cells is therefore defined as follows: Dch0ij<Dhi0ij(4)<Dhi0ij(1)<Dhh0ij.

Provided the maximum dose value which can be deposited in a volume comprising both tumoral and healthy cells is higher than the minimum target fraction dose (Dmt0j), then the irradiation fraction can successfully be carried out, killing the tumoral cells while sparing the healthy cells comprised within the irradiated volume within the predefined NTCP0i.

### Maximum and Minimum Doses

The treatment plan may fulfil a number of criteria. These may include DmtT=Σ Dmtj≥DmtT0, wherein DmtT is the total target dose required to kill tumoral cells in a volume during a fraction, and DmtT0 is the minimum target dose cumulated over the N fractions to kill all tumoral cells comprised within the peripheral surface. Criteria may also include Dmhij=Dchij+α Dhhij≤Dch0ij, wherein Dmhij is the equivalent healthy fraction dose and Dch0ij is the maximum conventional healthy fraction dose not to be exceeded during a fraction to preserve the healthy cells (cf. FIG. 4(c)). Criteria may also include DmhTi=Σ Dmhij=Σ(Dchij+α Dhhij)≤DchT0i, wherein DmhTi is the equivalent healthy dose and DchT0i is the maximum conventional healthy dose cumulated over the N fractions j not to be exceeded to preserve the healthy cell (cf. FIG. 7).

The criteria may also be expressed as Dhij=Dchij+Dhhij≤Dh0ij, wherein Dhij is the healthy fraction dose and Dh0ij is the maximum healthy fraction dose not to be exceeded during a fraction to preserve the healthy cells (cf. FIG. 5(a)), and DhTi=Σ Dhij=Σ(Dchij+Dhhij)≤DhT0i, wherein DhTi is the healthy dose cumulated over the N fractions j, and DhT0i is the maximum healthy dose cumulated over the N fractions j not to be exceeded to preserve the healthy cells.

The minimum target dose (DmtT0) must be exceeded to ensure that the treatment kills the tumoral cells comprised within the peripheral surface at the end of the N fractions j. If the tumoral cells belong to a same tissue or organ as healthy cells (3hi) located within or adjacent the peripheral surface, then the minimum target dose (DmtT0) would most probably be higher than the maximum conventional healthy dose (DchT0i) not to be exceeded in order to spare said healthy cells of same nature as the tumoral cells. This of course constitutes an apparently inescapable trap as it would therefore seem impossible to determine a dose sufficiently high to kill the tumoral cells and sufficiently low to spare the healthy cells adjacent to the tumoral cells. The minimum target dose (DmtT0) may be predefined by a medical practitioner.

As can be seen in FIG. 5(a), the maximum healthy fraction dose Dh0ij may be comprised between the maximum conventional fraction dose (Dch0ij) and the maximum high rate fraction dose (Dhh0ij), i.e., Dch0ij≤Dh0ij≤Dhh0ij, depending on the HDR proportion, xH=Dhhij/(Dchij+Dhhij). In one embodiment, the maximum healthy fraction dose Dh0ij may be expressed as follows, Dh0ij=Dch0ij+(1−α)Dhhij(xH), wherein α may or may not vary with xH, and Dhhij(xH) is the high rate healthy fraction dose at a value of xH such that Dmhij=Dch0ij.

Similarly, the maximum healthy dose DhT0i is comprised between the maximum conventional healthy dose (DchT0i) and the maximum high rate healthy dose (DhhT0i), i.e., DchT0i≤DhT0i≤DhhT0i, depending on the HDR proportion, xH. In one embodiment, DhT0i can be expressed as, DhT0i=DchT0i+(1−α)DhhTi(xH), wherein α may or may not vary with xH, and DhhTi(xH) is the high rate healthy dose at a value of xH such that DmhTi=DchT0i.

The maximum conventional healthy fraction dose (Dch0ij) and the maximum conventional healthy dose (DchT0i) which define the fraction dose and total cumulated dose deposited at CDR only, yielding a predefined NTCP0i not to be exceeded to preserve the healthy cells and which are used to define criteria, may be predefined by a medical practitioner or defined in the literature. For example, Table 1 lists values of DchT0i for a number of organs/tissues, for a value of NTCP0i=50% and diagnosed 5 years after the radiotherapy, extracted from Table 3 in Kehwar, J Cancer Res Ther., (September 2005), 1 (3), p. 170. These values are representative of what a medical practitioner could use as reference to define a value of DchT0i.

TABLE 1
Examples of DchT0i values for some tissues irradiated at CDR
only (Kehwar (2005))

| Healthy tissue | DchT0i | |
|---|---|---|
| (3hi) | (Gy) | End point |
| brain | 60 | Necrosis/infraction |
| heart | 50 | Pericarditis |
| larynx | 80 | Cartilage necrosis |
| Liver | 40 | Liver failure |
| Lung | 25 | Pneumonitis |
| Stomach | 65 | Ulceration/perforation |
| Eye lens | 18 | Cataract requiring intervention |
| Ear (Mid/Ext) | 65 | Chronic serious otitis |
| Oesophagus | 68 | Clinical stricture |
| perforation | | |
| Bladder | 79 | Symptomatic bladder contraction |
| volume | | |
| Colon | 55 | Obstruction/perforation/ulceration/fistula |
| Spinal cord | 69 | Myelitis/necrosis |
| (20 cm) | | |
| Femoral head | 65 | Necrosis |
| and neck | | |
| Optic | 65 | Blindness |
| nerve/optic | | |
| chiasma | | |
| Retina | 65 | Blindness |
| Rib cage | 65 | Pathologic fracture |
| Parotid | 46 | Xerostomia |
| Thyroid | 80 | Clinical thyroidis |

The maximum high rate healthy fraction dose (Dhh0ij) and the maximum high rate healthy dose (DhhT0i) which define the fraction dose and total cumulated dose deposited at HDR only, yielding the predefined NTCP0i not to be exceeded to preserve the healthy cells and which can be used in the present disclosure, can either be predefined by a medical practitioner or defined in the literature for given values of the HDR, respectively defined as Dhh0ij=k Dch0i and Dhh0Ti=k DchT0i, for given values of the HDR, wherein k is comprised between 1.2 and 1.5 (i.e., k=1.2 to 1.5), or defined by a pre-established curve of a Normal Tissue Complication Probability (NTCP) for the healthy tissue (3hi) as a function of dose and dose deposition rate as illustrated in FIGS. 4(a) to 4(c), and 7, and wherein Dhh0i may be defined as the dose having same NTCP as Dch0i.

For example, values of DhhT0i can be estimated from the values of DchT0i listed in Table 1 by multiplying them by a factor k=1.5. The corresponding results are listed in Table 2.

TABLE 2
Examples of DhhT0i values for some tissues irradiated at HDR
only DhhT0i = k DchT0i

| Healthy tissue | DchT0i | |
|---|---|---|
| (3hi) | (Gy) | End point |
| brain | 90 | Necrosis/infraction |
| heart | 75 | Pericarditis |
| larynx | 120 | Cartilage necrosis |
| Liver | 60 | Liver failure |
| Lung | 38 | Pneumonitis |
| Stomach | 98 | Ulceration/perforation |
| Eye lens | 27 | Cataract requiring intervention |
| Ear (Mid/Ext) | 98 | Chronic serious otitis |
| Oesophagus | 102 | Clinical stricture |
| perforation | | |
| Bladder | 119 | Symptomatic bladder contraction |
| volume | | |
| Colon | 83 | Obstruction/perforation/ulceration/fistula |
| Spinal cord | 104 | Myelitis/necrosis |
| (20 cm) | | |
| Femoral head | 98 | Necrosis |
| and neck | | |
| Optic | 98 | Blindness |
| nerve/optic | | |
| chiasma | | |
| Retina | 98 | Blindness |
| Rib cage | 98 | Pathologic fracture |
| Parotid | 69 | Xerostomia |
| Thyroid | 120 | Clinical thyroidis |

### Local Distribution of Dose Depositions at CDR and HDR

For a given irradiation equipment with limited capacity to deliver a given dose at a given ultra-high rate (HDR), the properties of the FLASH effect can be taken advantage of by selecting a volume of the target tissue (3t) to be treated in a fraction j and, depending on the presence and location of healthy cells relative to the volume, to optimize the HDR-proportion xH=Dhhij/(Dchij+Dhhij) of doses deposited at CDR and at HDR, respectively. In one embodiment wherein the charged particles are protons, all healthy tissues (3hi) located upstream from the peripheral surface are irradiated at conventional dose deposition rates only during the N fractions. This is made possible because of the dose deposition profile of proton beams, comprising a low deposition increasing locally to form a Bragg peak. This is represented graphically in FIGS. 1(b) and 1(c), wherein the healthy tissue (3hi) first traversed by the proton beam represented by a cylinder to take account of a width of the proton beam, receives a low dose, which can “safely” be deposited at CDR within the predefined value of NTCP0i. The following criteria may be fulfilled the healthy fraction dose (Dhij) deposited in said healthy tissues at each fraction j does not exceed the maximum conventional healthy fraction dose (Dch0ij), and the total healthy dose (DhTi) deposited in said healthy tissues at the end of the N fractions does not exceed the maximum conventional healthy dose (Dch0Ti).

A healthy cell is located upstream of the peripheral surface when a charged particles beam defined in the TPS traverses said healthy cell before penetrating the peripheral surface. Similarly, a healthy cell is located downstream of the peripheral surface when a charged particle beam defined in the TPS traverses said healthy cell only after exiting the peripheral surface. A dose is “safely” deposited onto a healthy cell if the corresponding NTCP≤NTCP0i.

On the other hand, all healthy tissues (3hi) located downstream of the peripheral surface can be irradiated at least partially, solely at ultra-high dose deposition rates, wherein, the healthy fraction dose (Dhij) deposited in said healthy tissues at each fraction j does not exceed the maximum high rate healthy fraction dose (Dh0ij), and the total healthy dose (DhTi) deposited in said healthy tissues at the end of the N fractions does not exceed the maximum conventional healthy dose (Dh0Ti).

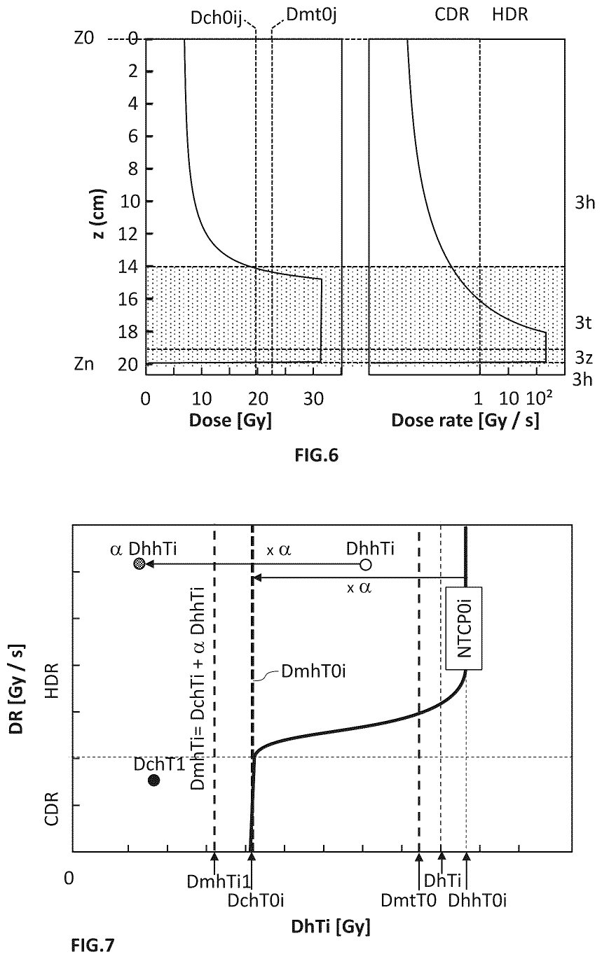

FIG. 6 illustrates an example of distribution in depth of penetration (z) of a proton beam of the dose (D) deposited in a fraction j, and of the dose deposition rate (DR). The dose (D) defines a portion of low doses deposited onto healthy cells of healthy tissues (3hi) located upstream of the tumoral cells of the target tissue (3t), followed by a SOBP of the type illustrated in FIGS. 1(a) and 1(c), spanning the whole depth of the target tissue (3t), falling abruptly downstream of the target tissue (3t). The cells located adjacent to the downstream end of the peripheral surface along the proton beam direction are the trickiest, because they potentially receive a high dose (D) of irradiation at the tail of the Bragg peak, and may comprise healthy cells mixed with tumoral cells in an uncertain zone (3z) located within the peripheral surface, or be formed of healthy tissues (3hi) outside of the peripheral surface. For this reason, the dose (D) may be deposited along the beam passage be deposited at varying deposition rates (DR), with deposition at CDR upstream of and in the upstream portion of the peripheral surface, increasing until reaching HDR as the beam penetrates deeper closer to and crosses the downstream end of the peripheral surface. The deposition rate must be at least partly at HDR when the dose (D) deposited in a volume exceeds the maximum conventional healthy fraction dose (Dch0ij) in case healthy cells are present in the volume. In the example of FIG. 6, healthy tissues can be exposed to doses higher than Dch0ij in particular in the uncertain zone (3z) located within a downstream portion of the peripheral surface, and also directly downstream of the peripheral surface. Such doses may be deposited at least partly at HDR to satisfy criteria.

### Uncertain Zone (3z)

The peripheral surface enclosing the tumoral cells of the target tissue (3t) may not form a clear interface separating tumoral cells inside thereof from healthy cells outside thereof, but may form instead an interphase defining an uncertain zone (3z) comprising both tumoral and healthy cells intermixed at different ratios. FIGS. 2(a), 2(b), and 6 show uncertain zones (3z) located in a downstream portion of the peripheral surface because, as discussed supra, they are the trickiest when irradiating with proton beams. It is clear, however, that such uncertain zones (3z) may be located anywhere along the peripheral surface, or even within the bulk of the target tissue (3t) in case there is an island of healthy cells enclosed within the peripheral surface, as shown in FIGS. 2(a) and 2(b). To reduce the undesirable side effects of an irradiation treatment, all healthy cells, even those included in an uncertain zone may be spared. These healthy cells are, however, particularly difficult to spare because they are exposed to a same irradiation dose as the tumoral cells, which must be at least equal to the minimum target fraction dose (Dmt0j). For this reason, to date most clinicians may favour certainty of destroying the tumoral cells, to the expenses of any healthy cells comprised within a same irradiation volume, rather than trying to spare the healthy cells with a risk of keeping tumoral cells active. In an embodiment, the TPS comprises the delivery to an entirety of the uncertain zone (3z) at the end of N fractions of a total uncertain dose (DuTi). If healthy cells are to be spared, then the maximum value of DuTi may be defined by the preservation of the healthy cells. In these conditions, we can stipulate that the total uncertain doses DuTi is equivalent to the total healthy dose (DhTi), and the following conditions apply to healthy cells comprised in an uncertain zone or any irradiated volume comprising healthy cells exposed to a same irradiation dose as the tumoral cells comprised within the irradiated volume, such as for example, but not restricted to, an island of healthy cells enclosed within the peripheral surface, as illustrated in FIGS. 2(a) and 2(b).

In this case, it may be that, as illustrated in FIG. 7, the total healthy dose (DhTi=DchTi+DhhTi) is larger than the minimum target dose (DmtT0), and lower than the maximum high rate healthy dose (DhhT0i) (i.e., DmtT0<DhTi<DhhT0i), wherein DchTi and DhhTi are the conventional and high rate healthy doses deposited at CDR and HDR, respectively, cumulated over the N fractions j and a total equivalent healthy dose (DmhTi=DchTi+α DhhTi) delivered at the end of the N fractions is lower than the maximum conventional healthy dose (DchT0i) of the healthy tissue (3hi) present in the uncertain zone (i.e., DmhTi<DchT0i), and an equivalent healthy fraction dose (Dmhij=Dchij+α Dhhij) delivered at each fraction j is lower than the maximum conventional healthy fraction dose (DchT0ij) of the healthy tissue (3hi) present in the uncertain zone, (i.e., DmhTi<DchT0i).

### Multi-Step Treatment Plan

In an embodiment of the disclosure, one or more fractions of the treatment plan include a first step of delivering a conventional dose at a conventional dose deposition rate (CDR) with a first charged particles beam or group of charged particles beams parallel to a first direction each formed by various beamlets. Referring to FIG. 1(a), this would correspond to the delivery in the first step of all the beamlets labelled “Dchij”. The conventional dose is such that a conventional target fraction dose (Dctj) is delivered at each or some of the one or more fractions to at least 90%; or, in some embodiments, at least 95%, or at least 99% of the tumoral cells of the target tissue (3t), and a conventional healthy fraction dose (Dchi) lower than the maximum conventional healthy fraction dose (Dch0ij) is delivered at each or some of the one or more fractions to the healthy tissues (3hi) surrounding and enclosed within the peripheral surface (i.e., Dchij<Dch0ij).

This first (conventional) step is followed or preceded by a second step of delivering a high rate dose at an ultra-high dose deposition rate (HDR) with a second charged particles beam or group of charged particles beams parallel to a second direction same as or different from the first direction. Referring to FIG. 1(a), this would correspond to the delivery in the second step of the beamlets labelled “Dhhij”. The high rate dose is such that a high rate target fraction dose (Dhtj) is delivered at each or some of the one or more fractions to the target tissue (3t) such that 100% of the tumoral cells of the target tissue (3t) have received a fraction dose at conventional and/or at ultra-high rates (CDR, HDR), and a high rate healthy fraction dose (Dhhij) is delivered at each or some of the one or more fractions to the healthy tissues (3hi) surrounding and within the peripheral surface, such that the equivalent healthy fraction dose (Dmhij=Dchij+α Dhhij) delivered at both conventional and ultra-high rates (CDR, HDR) to all healthy cells, does not exceed the maximum conventional healthy fraction dose (Dch0ij) (i.e. Dmhij≤Dch0ij).

The first step of conventional irradiation is like painting an area enclosed within a perimeter with a large brush, followed by painting the edges of the area with a thinner brush, to ensure that the area adjacent to and within the perimeter is fully coloured, while no colour is visible outside the perimeter. First and second steps may be carried out in any order.

To ensure that in a fraction j the healthy cells do not receive a healthy fraction dose (Dhii) cumulated over the first and second steps, which exceeds the maximum healthy fraction dose (Dh0ij), the treatment plan can ensure that in a same fraction the second charged particles beam or group of charged particles beams does not overlap with the first charged particles beam or group of charged particles beams in any healthy tissue (3hi), surrounding or within the peripheral surface.

### Single-Step Treatment Plan

In an alternative embodiment of the disclosure, one or more fractions of the treatment plan include a single charged particles beam (PB) or group of beams parallel to a first direction formed by beamlets, such that a conventional target fraction dose (Dctj) is delivered to at least 90%, or, in some embodiments, at least 95%, or at least 99% of the tumoral cells of the target tissue (3t), a conventional healthy fraction dose (Dchij) lower than the maximum conventional healthy fraction dose (Dch0ij) is delivered to the healthy tissues (3hi) surrounding and enclosed within the peripheral surface (i.e., Dchij<Dch0ij), a high rate target fraction dose (Dhtj) is delivered at each or some of the one or more fractions to the target tissue (3t) such that 100% of the tumoral cells of the target tissue (3t) have received a fraction dose at conventional and/or at ultra-high rates (CDR, HDR), and a high rate healthy fraction dose (Dhhij) is delivered at each or some of the one or more fractions to the healthy tissues (3hi) surrounding and within the peripheral surface, such that the equivalent healthy fraction dose (Dmhij=Dchij+α Dhhij) delivered at both conventional and ultra-high rates (CDR, HDR) to all healthy cells, does not exceed the maximum conventional healthy fraction dose (Dch0ij) (i.e. Dmhij≤Dch0ij).

The difference of the present single-step treatment plan with the multi-step treatment plan discussed supra, is that all beamlets of a fraction j are delivered within a single irradiation step instead of two steps for the multi-step treatment plan. Referring to FIG. 1(a), this means that in a single step, the beamlets labelled “Dchij” and the beamlets labelled “Dhhij” are all delivered within the same irradiation beam which is modulated to vary the doses deposited as a function of penetration depth (z).

FIG. 3 shows N fractions, each fraction comprising deposition of a conventional fraction dose (Dchij) (shaded columns) and of a high rate fraction (Dhhij) (white columns). The HDR-proportion, xH=Dhhij/(Dchij+Dhhij) can vary substantially from one fraction to the other. The respective conventional and high rate fraction doses may fulfil the following criteria. First, the total target dose (DmtT) cumulated over the N fractions (DmtT=Σ Dmtj=DctT+DhtT=Σ(Dctj+Dhtj)) must be larger than the predefined minimum target dose (DmtT0), (i.e., DmtT>DmtT0), wherein DctT and DhtT are the conventional and high rate target doses deposited at CDR and HDR, respectively, cumulated over the N fractions j, and second, the total equivalent healthy dose (DmhTi) cumulated over the N fractions (DmhTi=Σ Dmhij=DchTi+α DhhTi=Σ(Dchij+α Dhhij)) must be lower than the predefined maximum conventional healthy dose (DchT0i) (i.e., DmhTi>DchT0i).

According to the multi-step treatment plan, the conventional dose (Dchij) at each fraction j (cf., shaded columns in FIG. 3) is deposited in the first step, followed by the second step of depositing the high rate dose (Dhhij). According to the single-step treatment plan, all beamlets depositing doses at both CDR and HDR are sent in a single irradiation, with the properties (including deposition rate) of the successive beamlets changing with depth (z) of the corresponding Bragg peaks.

A treatment plan for treatment with charged particles beams, for instance with proton beams, of a target tissue comprising tumoral cells must fulfil predefined clinical criteria for ensuring that at the end of the treatment, the tumoral cells are killed, while sparing the healthy cells adjacent to the tumoral cells. The criteria may include killing of tumoral cells, wherein the total target dose (DmtT) delivered to the whole volume comprised within the peripheral surface must exceed the minimum target dose (DmtT0), i.e., DmtT=Σ Dmtj≥DmtT0. The criteria may also sparing of healthy cells wherein the healthy fraction dose (Dhij) deposited in one fraction j cannot exceed the maximum healthy fraction dose (Dh0ij), i.e., Dhij=Dchij+Dhhij≤Dh0ij, and the healthy dose cumulated over the N fractions j (DhTi) cannot exceed the maximum healthy dose cumulated over the N fractions j (DhT0i), i.e., DhTi=Σ Dhij=Σ(Dchij+Dhhij)≤DhT0i.

If healthy cells are adjacent to tumoral cells, the healthy cells are likely to receive a same dose as the tumoral cells. If the dose received is sufficient for killing the tumoral cells, it is likely that it is also sufficient for killing the healthy cells, which is of course undesirable. The present disclosure takes advantage of the FLASH effect observed at ultra-high dose deposition rates (HDR) by combining in the treatment plan conventional irradiation at CDR and FLASH-irradiation of specific volumes at HDR.

The criterion may be fulfilled by ensuring that the total target dose DmtT=DctT+DhtT is greater than the minimum target dose (DmtT0).

The criterion may be fulfilled by ensuring that the healthy fraction dose (Dhij=Dchij+Dhhij) is lower than the maximum healthy fraction dose (Dh0ij). This criterion is satisfied if the equivalent healthy fraction dose (Dmhij=Dchij+α Dhhij) does not exceed the maximum conventional healthy fraction dose (Dch0ij), i.e., Dmhij=Dchij+α Dhhij≤Dch0ij. The equivalent healthy fraction dose (Dmhij) is the dose deposited at least partly at HDR yielding a same value of complication probability (NTCP0i) as if the same dose had been deposited at CDR only.

The criterion may be fulfilled by ensuring that, the total healthy dose (DhTi=DchTi+DhhTi) is lower than the maximum total healthy dose (DhT0i). This criterion is satisfied if the equivalent healthy dose (DmhTi=DchTi+α Dhhij) does not exceed the maximum conventional healthy fraction dose (DchT0i, i.e., Dmhij=Dchij+α DhhTi≤DchT0i. The equivalent total healthy dose (DmhTi) is the cumulated dose deposited at least partly at HDR over the N fractions, yielding a same value of complication probability (NTCP0i) as if the same dose had been deposited at CDR only.

With the above treatment plan, if the healthy fraction dose (Dhij) deposited onto the healthy cells in a fraction j is larger than both the minimum target dose (DmtT0) required for killing the tumoral cells and the healthy cells comprised within the volume radiated in fraction j, a trap is opened by combining deposition at CDR with deposition at HDR to profit of the FLASH effect. The trap is open even in the worst-case scenario wherein both tumoral and healthy cells receive a same radiation dose (Dmtij=Dhij), thus fulfilling both Criteria (C1) and (C2.1). The same conclusion applies to the full treatment at the end of N fractions, such that if the total healthy fraction (DhTi) deposited onto the healthy cells after N fractions is deposited at least partly at HDR, it can be larger than the minimum target dose (DmtT0) required for killing the tumoral cells within the peripheral surface, and lower than the maximum total healthy dose (DhT0i) required for sparing the healthy tissues (3hi). Here again the apparently inescapable trap is opened even in the worst-case scenario wherein both tumoral and healthy cells receive a same radiation dose, thus fulfilling both Criteria (C1) and (C2.2). The cases wherein Dhij=Dmtj and, after N fractions, DhTi=DmTt is referred to as “worst case scenario,” because in most cases, if the healthy cells are outside the peripheral surface, the treatment plan attempts to reduce the dose (Dhij) delivered to the healthy cells and to maximize the dose (Dmtj) delivered to the tumoral cells, so that Dhij<Dmtj and, after N fractions, DhTi<DmTt.

The treatment plan generated by the TPS of the present disclosure may be implemented in a method for treating with charged particles beams, for instance, with proton beams, a target tissue (3t) comprising tumoral cells enclosed within a peripheral surface, which is surrounded by healthy cells and/or encloses also healthy cells, wherein the healthy cells form healthy tissues (3hi), wherein the subscript i designates the type of healthy tissues (3hi), wherein the method consists of N fractions of irradiation, with N≥1. The plan may comprise fulfilling the following criteria. First, at the end of the N fractions, all tumoral cells of the target tissue (3t) must have received a total target dose (DmtT) equal to the sum of target fraction doses (Dmtj) received at each fraction (i.e., DmtT=Σ Dmtj), which is at least equal to a minimum target dose (DmtT0) for killing the tumoral cells, i.e., DmtT=Σ Dmtj≥DmtT0. Second, at the end of the N fractions, all healthy cells (3hi) of the healthy tissues surrounding or enclosed within the peripheral surface must have received a total healthy dose (DhTi) equal to the sum of the healthy fraction doses (Dhij) received at each fraction j (i.e., DhTi=Σ Dhij), such that the healthy fraction dose (Dhij) received at each fraction j does not exceed a predefined threshold for preserving the healthy cells at the end of the fraction j, and the total healthy doses (DhTi) received and cumulated at the end of the N fractions does not exceed a predefined threshold for preserving the healthy cells at the end of the treatment of N fractions.

Further, the method may combine N fractions including, irradiation of volumes including tumoral cells and/or healthy cells at a conventional dose rate (CDR), defined as a dose rate, CDR<1 Gy/s, and/or irradiation of volumes including tumoral cells and/or healthy cells at an ultra-high dose rate (HDR), defined as a dose rate, HDR≥1 Gy/s, wherein the total target dose (DmtT) delivered to and cumulated in a volume of the target tissue (3t) at the end of N fractions is defined to fulfill criterion (C1) as a sum over the N fractions j, DmtT=Σ Dmtj=Σ(Dctj+Dhtj)≥DmtT0, of target fraction doses (Dmtj) delivered to the target tissues (3t) at each fraction, wherein the target fraction dose (Dmtj) delivered during a fraction j is defined as a sum of, conventional target doses (Dctj) delivered to that volume at conventional dose rates (CDR) during the fraction j, and of high rate target doses (Dhtj) delivered to that volume at ultra-high dose rates (HDR) during the fraction j.

Further, the healthy fraction dose (Dhij) delivered to a volume of healthy cells of a healthy tissue (3i) during a fraction j is equal to a sum, Dhij=Dchij+Dhhij, of a conventional healthy fraction dose (Dchij) delivered to that volume at a conventional dose rate (CDR), and of a high rate healthy fraction dose (Dhhij) delivered to that volume at ultra-high dose rate (HDR), an equivalent healthy fraction dose (Dmhij) defined as a sum Dmhij=(Dchij+α Dhhij), of the conventional healthy fraction dose (Dchij) and a product of an equivalence coefficient (α) and of the high rate healthy fraction dose (Dhhij), does not exceed a maximum equivalent healthy fraction dose (Dmh0ij), for preserving the healthy cells after each fraction j (i.e., Dmhij≤Dmh0ij), and thus fulfilling criterion (C2.1), wherein the maximum equivalent healthy fraction dose (Dmh0ij) is smaller than or equal to a maximum healthy conventional fraction dose (Dch0ij) corresponding to a dose deposited onto a healthy cell at a conventional dose rate (CDR) only and yielding a predefined probability of complication (NTCP0i) of the healthy cell (3hi) (i.e., Dmh0ij≤Dch0ij) a total equivalent healthy dose (DmhTi) delivered to and cumulated in a volume of a healthy tissue (3hi) at the end of N fractions is defined as a sum over the N fractions j, DmhTi=Σ Dmhij=Σ(Dchij+α Dhhij) with α≤1, or, in some embodiments, α>0.5, of the equivalent healthy fraction doses (Dmhij), wherein the total equivalent healthy dose (DmhTi) does not exceed a maximum equivalent healthy dose (DmhT0i), for preserving the healthy cells at the end of the N fractions (i.e., DmhTi≤DmhT0i), and thus fulfilling criterion (C2.2), wherein the maximum equivalent healthy dose (DmhT0i) is smaller than or equal to a maximum healthy conventional dose (DchT0i) corresponding to a dose deposited onto a healthy cell at a conventional dose rate (CDR) only and yielding a predefined probability of complication (NTCP0i) of the healthy cell (3hi) (i.e., DmhT0i≤DchT0i).

Further, the equivalent healthy fraction dose (Dmhij) and the total equivalent healthy dose (DmhTi) are equivalent doses yielding a same or lower probability of complication of a healthy cell (3hi) (NTCP0) as if the healthy fraction dose (Dhij) and the total healthy dose (DhTi) had, respectively, been deposited onto the healthy cell at a conventional dose rate (CDR) only.

What is claimed is:

1. A system for treating a target tissue with a charged particle beam, wherein the target tissue includes tumoral cells enclosed within a peripheral surface and the peripheral surface is surrounded by or encloses healthy cells of healthy tissue, the system comprising a computer configured to:
receive a total target dose to be delivered to the tumoral cells of the target tissue, the total target dose being greater than or equal to a minimum target dose for killing the tumoral cells;
receive a fraction number (N) of irradiation fractions for treating and for delivering the total target dose to the target tissue, the fraction number (N) being an integer and equal to or greater than one;
receive a maximum healthy fraction dose and a maximum healthy total dose deposited onto the healthy cells at a conventional dose deposition rate (CDR), wherein the maximum healthy fraction dose and the maximum healthy total dose are not exceeded to preserve the healthy cells within or adjacent to the target tissue;
receive an equivalence coefficient (α) for the healthy tissue, the equivalence coefficient (α) being a correspondence factor between a complication probability of a healthy tissue caused by a dose deposited at the conventional dose deposition rate (CDR) and at an ultra-high dose deposition rate (HDR) in accordance with a normal tissue complication probability (NTCP) curve, the NTCP curve being associated with specific tissues traversed by the charged particle beam, dose values, and dose deposition rates, wherein the CDR is less than 1 gray per second, and the HDR is greater than or equal to 1 gray per second;
divide the total target dose into N target fraction doses to be respectively deposited during N irradiation fractions; and
determine, for each of the N irradiation fractions, a ratio of a target fraction dose to be deposited at the CDR and a complementary ratio of the target fraction dose to be deposited at the HDR based on an equivalent fraction dose, the maximum healthy fraction dose, a total equivalent dose, and the maximum healthy total dose,
wherein:
the equivalent fraction dose is defined as a sum of a proportion of a fraction dose deposited at the CDR and a product of multiplying the equivalence coefficient (α) by a complementary proportion of a fraction dose deposited at the HDR onto the healthy cells,
the equivalent fraction dose deposited onto the healthy cells traversed by the charged particle beam is lower than or equal to the maximum healthy fraction dose,
the total equivalent dose is defined as a sum of N equivalent fraction doses and the total equivalent dose deposited onto the healthy cells traversed by the charged particle beam during all of the N irradiation fractions is lower than or equal to the maximum healthy total dose, and wherein
a sum of target fraction doses deposited at the CDR and HDR over the N irradiation fractions is greater than or equal to the total target dose.

2. The system according to claim 1, wherein the healthy cells are irradiated with a fraction dose in one of the N irradiation fractions, wherein during the one of the N irradiation fractions:
the healthy cells are irradiated only at the CDR, wherein the fraction dose deposited onto the healthy cells at the HDR is zero and the equivalent fraction dose deposited onto the healthy cells is equal to the fraction dose deposited at the CDR, and equal to or less than the maximum healthy fraction dose; or
the healthy cells are irradiated only at the HDR, wherein the fraction dose deposited onto the healthy cells at the CDR is zero and the equivalent fraction dose deposited onto the healthy cells is equal to the product of the equivalence coefficient (α) and the fraction dose deposited at the HDR, and is equal to or less than the maximum healthy fraction dose, or
the healthy cells are irradiated at the CDR and HDR, wherein the equivalent fraction dose deposited onto the healthy cells is equal to the sum of a portion of the fraction dose deposited at the CDR and the product of the equivalence coefficient (α) and a portion of the fraction dose deposited at the HDR onto the healthy cells, and is equal to or less than the maximum healthy fraction dose.

3. The system according to claim 1, wherein the healthy cells are irradiated with a fraction dose in one of the N irradiation fractions, wherein:
the equivalence coefficient (α) is a constant and equal to an inverse of a number (k), the number (k) being between 1.2 and 1.5, or
the equivalence coefficient (α) depends on an HDR proportion of the fraction dose deposited onto the healthy cells and is between the inverse of the number (k) and 1, wherein:
when the HDR proportion is equal to 0, the fraction dose is deposited at the CDR, and the equivalence coefficient (α) is equal to 1; and
when the HDR proportion is equal to 1, the fraction dose is deposited at the HDR, the equivalence coefficient (α) is equal to the inverse of the number (k).

4. The system according to claim 3, wherein the equivalence coefficient (α) is a constant that is less than 0.9.

5. The system according to claim 4, wherein the equivalence coefficient (α) is a constant that is less than 0.85.

6. The system according to claim 5, wherein the equivalence coefficient (α) is a constant that is less than 0.7.

7. The system according to claim 2, wherein:
the maximum healthy fraction dose is a first predefined dose,
the maximum healthy total dose is a second predefined dose,
the equivalent fraction dose deposited onto the healthy cells is a calculated dose based on the equivalence coefficient (α), a proportion of the fraction dose deposited at the CDR, and a proportion of the fraction dose deposited at the HDR, and
the equivalent total dose is a calculated dose based on the fraction number (N) and the equivalent fraction doses of the N irradiation fractions.

8. The system according to claim 2, wherein:
the maximum healthy fraction dose is a first predefined dose,
the maximum healthy total dose is a second predefined dose,
the equivalent fraction dose deposited onto the healthy cells only at the HDR is equal to or less than the maximum healthy fraction dose if the fraction dose deposited onto the healthy cells is equal to or less than the maximum healthy fraction dose multiplied by a number between 1.2 and 1.5, and
the equivalent total dose deposited onto the healthy cells only at the HDR is equal to or less than the maximum healthy total dose if a sum of fraction doses deposited onto the healthy cells during the N irradiation fractions is equal to or less than the maximum healthy total dose multiplied by the number between 1.2 and 1.5.

9. The system according to claim 1, wherein:
a fraction dose deposited at the CDR has a first normal tissue complication probability (NTCP) on the tumoral and healthy cells, and
the fraction dose deposited at the HDR has the first NTCP on the tumoral cells and a second NTCP on the healthy cells, the second NTCP being lower than the first NTCP.

10. The system according to claim 1, wherein:
an upstream portion of the healthy cells is located upstream from the peripheral surface and penetrated by the charged particle beam before the peripheral surface is penetrated thereby,
the upstream portion of the healthy cells is only irradiated at the CDR,
the equivalent fraction dose deposited in the upstream portion of the healthy cells in each of the N irradiation fractions does not exceed the maximum healthy fraction dose, and
the total equivalent dose deposited in the upstream portion of the healthy cells is a sum of N equivalent fraction doses deposited therein in the N irradiation fractions, the total equivalent dose deposited in the upstream portion of the healthy tissue not exceeding the maximum healthy total dose.

11. The system according to claim 1, wherein:
a downstream portion of the healthy cells is located downstream of the peripheral surface and penetrated by the charged particle beam after the peripheral surface is penetrated thereby,
the downstream portion of the healthy cells is only irradiated at the HDR,
the equivalent fraction dose deposited in the downstream portion of the healthy cells in each of the N irradiation fractions does not exceed a maximum healthy fraction dose, and
the total equivalent dose deposited in the downstream portion of the healthy cells after the N irradiation fractions does not exceed the maximum healthy total dose.

12. The system according to claim 1, wherein the charged particle beam includes a first charged particle beam and a second charged particle beam, and the healthy cells are irradiated with a fraction dose in each of the N irradiation fractions, wherein:
a dose is delivered at the CDR by the first charged particle beam parallel to a first direction such that:
a target fraction dose in at least one of the N irradiation fractions is delivered at the CDR to at least 90%, 95%, or 99% of the tumoral cells of the target tissue, and
a fraction dose in at least one of the N irradiation fractions is delivered at the CDR to the healthy cells and is less than the maximum healthy fraction dose; and
a dose is delivered at the HDR by the second charged particle beam parallel to a second direction such that:
a target fraction dose in at least one of the N irradiation fractions is delivered at the HDR to the target tissue such that all of the tumoral cells of the target tissue receive at least one of the target fraction doses at the CDR or at the HDR, and
a fraction dose in at least one of the N irradiation fractions is delivered at the HDR to the healthy cells, and the equivalent fraction dose deposited onto the healthy cells does not exceed the maximum healthy fraction dose.

13. The system according to claim 12, wherein the second charged particle beam does not overlap with the first charged particle beam in the healthy cells.

14. The system according to claim 1, wherein the charged particle beam is parallel to a direction, wherein:
a target fraction dose in at least one of the N irradiation fractions is delivered at the CDR to at least 90%, 95%, or 99% of the tumoral cells of the target tissue;
a fraction dose in at least one of the N irradiation fractions is delivered at the CDR to the healthy cells and is less than the maximum healthy fraction dose;
a target fraction dose in at least one of the N irradiation fractions is delivered at the HDR to the target tissue such that all of the tumoral cells of the target tissue receive at least one of the target fraction doses delivered at the CDR or at the HDR; and
a fraction dose in at least one of the N irradiation fractions is delivered at the HDR to the healthy cells such that the equivalent fraction dose deposited onto the healthy cells does not exceed the maximum healthy fraction dose.

15. The system according to claim 1, wherein:
the peripheral surface encloses a zone including a portion of the tumoral cells and a portion of the healthy cells;
in each of the N irradiation fractions, the equivalent fraction dose for the zone is less than the maximum healthy fraction dose; and
at the end of the N irradiation fractions, a total dose deposited onto the portion of the tumoral cells and the portion of the healthy cells in the zone is greater than the total target dose for killing the tumoral cells of the zone, and the total equivalent dose deposited onto the portion of the tumoral cells and the portion of the healthy cells in the zone is lower than the maximum healthy total dose.

16. The system according to claim 1, wherein the maximum healthy total dose deposited onto healthy cells at the CDR is:
60 grays for a brain tissue,
50 grays for a heart tissue,
80 grays for a larynx tissue,
40 grays for a liver tissue,
25 grays for a lung tissue,
65 grays for a stomach tissue, or
18 grays for an eye lens tissue.

17. The system according to claim 16, wherein a maximum dose, deposited onto healthy cells at the HDR and yielding a total equivalent dose equal to the maximum healthy total dose deposited onto the healthy cells at the CDR, is:
90 grays for a brain tissue,
75 grays for a heart tissue,
120 grays for a larynx tissue,
60 grays for a liver tissue,
38 grays for a lung tissue,
98 grays for a stomach tissue, or
27 grays for an eye lens tissue.

18. The system according to claim 1, wherein the charged particle beam is a proton beam delivered by single scattering, double scattering, or pencil beam scanning.

19. A method for treating a target tissue with a charged particle beam, wherein the target tissue includes tumoral cells enclosed within a peripheral surface and the peripheral surface is surrounded by or encloses healthy cells of healthy tissue, the method comprising:
receiving a total target dose to be delivered to the tumoral cells of the target tissue, the total target dose being greater than or equal to a minimum target dose for killing the tumoral cells;
receiving a fraction number (N) of irradiation fractions for treating and for delivering the total target dose to the target tissue, the fraction number (N) being an integer and equal to or greater than one;
receiving a maximum healthy fraction dose and a maximum healthy total dose deposited onto the healthy cells at a conventional dose deposition rate (CDR), wherein the maximum healthy fraction dose and the maximum healthy total dose are not exceeded to preserve the healthy cells within or adjacent to the target tissue;
receiving an equivalence coefficient (α) for the healthy tissue, the equivalence coefficient (α) being a correspondence factor between a complication probability of a healthy tissue caused by a dose deposited at the conventional dose deposition rate (CDR) and at an ultra-high dose deposition rate (HDR) in accordance with a normal tissue complication probability (NTCP) curve, the NTCP curve being associated with specific tissues traversed by the charged particle beam, dose values, and dose deposition rates, wherein the CDR is less than 1 gray per second, and the HDR is greater than or equal to 1 gray per second;
dividing the total target dose into N target fraction doses to be respectively deposited during N irradiation fractions; and
determining, for each of the N irradiation fractions, a ratio of a target fraction dose to be deposited at the CDR and a complementary ratio of the target fraction dose to be deposited at the HDR based on an equivalent fraction dose, the maximum healthy fraction dose, a total equivalent dose, and the maximum healthy total dose,
wherein:
the equivalent fraction dose is defined as a sum of a proportion of a fraction dose deposited at the CDR and a product of multiplying the equivalence coefficient (α) by a complementary proportion of a fraction dose deposited at the HDR onto the healthy cells,
the equivalent fraction dose deposited onto the healthy cells traversed by the charged particle beam is lower than or equal to the maximum healthy fraction dose,
the total equivalent dose is defined as a sum of N equivalent fraction doses and the total equivalent dose deposited onto the healthy cells traversed by the charged particle beam during all of the N irradiation fractions is lower than or equal to the maximum healthy total dose, and wherein
a sum of target fraction doses deposited at the CDR and HDR over the N irradiation fractions is greater than or equal to the total target dose.
